(19) United States
(12) Patent Application Publication
(10) Pub. No.: US 2024/0180750 A1
(43) Pub. Date: Jun. 6, 2024

(54) HEADSET

(71) Applicant: CLUISTROM3D LIMITED, Dunfermline (GB)

(72) Inventor: Neil MCINTOSH, Dunfermline (GB)

(73) Assignee: CLUISTROM3D LIMITED, Dunfermline (GB)

(21) Appl. No.: 18/574,509

(22) PCT Filed: Jul. 4, 2022
(86) PCT No.: PCT/GB2022/051713

§ 371 Date: Dec. 27, 2023

(30) Foreign Application Priority Data
Jul. 2, 2021 (GB) 2109630.0

(51) Int. Cl. A61F 11/14; H04R 1/10

(52) U.S. Cl. CPC A61F 11/145; H04R 1/1083; H04R 2460/15

(57) ABSTRACT

A seal verification module for a headset ear cup, a headset comprising such a module (e.g. the module being integral with, recessed into or mounted in/on the car cup), a method for retrofitting the seal verification module to a headset, and a method for verifying that a headset comprising an ear cup is correctly situated on a user's head such that the ear cup forms an acoustic seal around the user's ear. The seal verification module comprising an indicator configured to provide an indication in response to being activated; and a pressure differential detector configured to detect a pressure differential between an internal side of the seal verification module and an external side of the seal verification module, and to activate the indicator in response to the detected pressure differential reaching or exceeding a predetermined pressure differential associated with verifying the headset ear cup being correctly situated.

### FIELD

This relates to a headset, for example an ear defender muff type headset, and a pressure sensor arrangement for verifying correct situation of the headset over a user's ears.

### BACKGROUND

Ear defender muff type headsets are used for hearing protection and for communication in a variety of industrial settings. Muff type headsets are usually recommended for use in high noise environments. As used herein, the term “headset” may refer to devices that engage a user's ears to provide noise protection for the user. Headsets typically include two rigid noise-shielding cups for enclosing the ears. Each ear cup has a rigid flange secured to the rim of the cup for supporting cushions made of a soft material which are intended to conform to the wearers head to form acoustical seals between the cups and the head around the ears. A head band adjustably connects the two ear cups.

The human audio frequency band is generally 20 Hz to 20 kHz, however, some frequency sub-bands, such as 63 Hz to 8 kHz within the human audio frequency band are of special interest for hearing protection. Exposure to excessive sound levels can cause damage in two ways, mechanical trauma and sensorineural hearing loss.

A mechanical hearing loss indicates there is a problem with the mechanism that conducts sound from the environment to the inner ear. Problems in the external auditory canal (outer-ear), ear drum or the bones of hearing (the middle ear) may cause a conductive loss. This type of loss can often be corrected by medication or surgery. If it cannot be corrected, the individual can usually do well with a hearing aid.

A sensorineural hearing loss indicates a problem in the organs or nerves of hearing. There, may be damage to the cochlea, auditory nerve, or the auditory centres of the brain. An individual with sensorineural hearing loss may benefit from a hearing aid, cochlear implant, communication therapies or other medical management depending on the degree or cause of the loss.

Noise induced hearing loss (NIHL) in the workplace is a well-documented phenomenon that can cause physical and psychological problems to those afflicted as well as economic damage to workplaces. Noise induced hearing loss is a gradual process and is often not noticed until after damage is done. During the last few decades, hearing specialists have become increasingly aware that noisy workplace environments have the potential to produce NIHL.

Industrial noise can damage the hearing of personnel, and breach occupational health and safety legislation. This noise can be defined as the high-level sound produced by various noise working environments. Although NIHL has been a known problem for some time, the combination of ever louder environments and tightening noise exposure legislation is impacting on what is deemed to be safe working conditions.

Management of NIHL within the workplace is often confounded by a worker's need to clearly perceive their acoustic environment in order to safely carry out their job to a satisfactory level. This is especially true within noisy environments where users who require protection due to often working in hazardous noise levels are unable to adequately protect their hearing due to the need to hear themselves and their colleagues with great clarity. Additionally, headsets are often worn improperly either willingly due to user preferences that are at variance with best safety practices, or unwillingly due to eyeglass stems, jewellery, or other accessories worn by the user which may impact the effectiveness of the sound-resistant seal.

Health and Safety Executive (HSE) sets standards for hearing protection in high-noise environments, such as factories and flight lines. Personal Protective Equipment (PPE) Directive adopted by the European Council in 1989 was superseded in April 2018 by a new PPE Regulation (EU)2016/425. The new regulation was created to enhance consumer safety and ensure fair competition between companies who provide PPE for their sector. Regulatory changes include moving Hearing Protection from Category II to Category III. This category exclusively includes the risk that harmful noise may cause very serious consequences such as irreversible damage to health. Health and Safety officers, from April 2018, must make sure that the hearing protection products used by employees comply with the Personal Protective Equipment (PPE) Regulation (EU)2016/425. European standards have high status globally for being effective in setting performance levels and are used in many countries where they do not have their own product standards. Hearing protection products commonly conform to these standards accordingly and hold EU Type Examination certification.

Furthermore, the Control of Noise at Work Regulations 2005 came into force for all industry sectors in Great Britain on 6 Apr. 2006. The aim of the Noise Regulations is to ensure that workers' hearing is protected from excessive noise at their place of work, which could cause noise induced hearing loss and/or tinnitus.

The sound pressure level at which employers must assess the risk to workers' hearing health is 80 decibels. Hearing protection must be made available to personnel upon request. The sound pressure level, as a daily or weekly average exposure, at which employers by Law must provide hearing protection and install its compulsory use is 85 decibels. Under United Kingdom legislation, an employer is bound to take action if noise levels go beyond these benchmarks. Noise in the workplace can routinely reach levels in the potentially hazardous range of 100 dBA. Therefore, the noise exposure can clearly exceed the nominal benchmark of 85 dBA.

The acoustic seal formed by a headset between the cups and the head around the ears provides the performance characteristics of the hearing protection.

If a hearing defender is placed over each ear the user has no way of confirming if an acoustic seal is being obtained because of different contributing factors including but not limited to head and facial hair, the wearing of eye protection, or other factors mentioned above, and so the user becomes reliant on the hearing defender manufacturer lab tested results and performance. Knowing this, HSE de-rates hearing protection devices from the maximum technical capability of the actual hearing protection headset to a lower rating to take improper fit into consideration. Consequently, hearing protection devices are commonly over-designed and over-constructed in order to qualify for use after the de-rating. Such overcompensation for improperly used hearing protection increases costs of providing hearing protection. Therefore, a need exists for a cost-effective hearing protection headset that can verify whether it is being properly worn.

An object and feature of an embodiment of the present invention is to overcome the above-mentioned problems and fulfill the above-mentioned needs.

### SUMMARY

According to a first aspect there is provided a seal verification module for a headset ear cup, the module comprising:
an indicator configured to provide an indication in response to being activated;
a pressure differential detector configured to detect a pressure differential between an internal side of the seal verification module and an external side of the seal verification module. The pressure differential detector may be configured to configured to activate the indicator in response to the detected pressure differential reaching or exceeding a predetermined pressure differential, for example associated with verifying the headset ear cup being correctly situated to form an acoustic seal against a surface.

The pressure differential detector may be configured to detect the differential in pressure between an internal side of the seal verification module and an external side of a seal verification module. Herein the internal side of the seal verification module refers to the side of the seal verification module that faces the inside of a headset ear cup in use. Herein the external side of the seal verification module refers to the side of the seal verification module that faces the outside of the headset ear cup in use. The predetermined pressure differential may be a predetermined differential in pressure between the internal side of the seal verification module and the external side of the seal verification module. In particular, the predetermined pressure differential may be a predetermined amount that the pressure on the internal side of the seal verification module is greater than the pressure on the external side of the seal verification module. In use, the pressure differential across the pressure differential detector may reach or exceed the predetermined pressure differential due to a change of pressure on the internal side of the seal verification module, in particular an increase of pressure on the internal side of the seal verification module. The pressure differential across the pressure differential detector may reach or exceed the predetermined pressure differential when the pressure on the internal side of the seal verification module changes by at least the predetermined amount, in particular when the pressure on the internal side of the seal verification module increases by at least the predetermined amount.

The pressure differential detector may be configured to detect an air pressure differential across the pressure differential detector. The pressure differential detector may be configured to detect ultra-low pressure differentials across the pressure differential detector. The predetermined pressure differential may be an ultra-low pressure differential. Herein, the term “ultra-low pressure differential” is used to refer to pressure differentials above 700 Pa (7 Millibar), for example in the region of 700 Pa to 10,000 Pa.

The pressure differential detector may generally act as a switch. In particular, the pressure differential detector may act as a DC switch. The pressure differential detector functioning as a switch may control the indicator. The pressure differential detector functioning as a switch may be biased towards the ‘off’ condition. When the pressure differential detector is in the ‘off’ condition the indicator may be off. The pressure differential detector functioning as a switch may change to the ‘on’ condition in response to the pressure differential across the pressure differential detector reaching or exceeding the predetermined pressure differential. The pressure differential across the pressure differential detector may reach or exceed the predetermined pressure differential due a change, e.g. increase, in pressure on the internal side of the seal verification module. Accordingly, the pressure differential detector functioning as a switch may change from the ‘off’ condition to the ‘on’ condition in response to the pressure on the internal side of the seal verification module changing, e.g. increasing, by at least a predetermined amount. When the pressure differential detector is in the ‘on’ condition the indicator may be activated to provide an indication to verify the headset ear cup being correctly situated to form an acoustic seal against a surface. Where the headset ear cup is not correctly situated to form an acoustic seal, the pressure differential across the seal detection module may not reach the predetermined pressure differential and the pressure differential detector may remain in the ‘off’ condition and the indicator may remain off.

In use, the seal verification module may be arranged with the headset ear cup such that a pressing action on the headset ear cup when it is correctly situated to form an acoustic seal against a surface, e.g. a user's head, will cause the pressure differential across the pressure differential detector to reach or exceed the predetermined pressure differential, and thus activate the indicator.

Beneficially, the present invention provides a module that can selectively indicate when a hearing protection headset is fitted correctly on a user. The indicator may only be activated if the necessary acoustic seal between the headset ear cup and the user's head is obtained. The module has benefits because it is efficient, inexpensive and convenient.

The indicator may comprise a light indicator, for example a Light Emitting Diode (LED), to provide visual verification of the seal. The LED may comprise an associated resistor. The indicator may alternatively or additionally comprise a sound indicator, for example a buzzer, to provide audible verification of the seal. Where the indicator comprises a sound indicator, the indicator may comprise a sound transfer opening to provide sound from the sound indicator to a contact face between the seal verification module and the ear cup or, when the seal verification module is integral with the ear cup, to the external side of the ear cup. The indicator may alternatively or additionally comprise a display.

The pressure differential detector may comprise a diaphragm. One side of the diaphragm may be exposed to the external side of the seal verification module. The other side of the diaphragm may be exposed to the internal side of the seal verification module. In use, one side of the diaphragm may be exposed to atmospheric pressure (herein intended as any pressure, e.g. air pressure, acting on the outside of the headset ear cup, not strictly atmospheric pressure of approximately 1 bar) and the other side of the diaphragm may be exposed to the pressure, e.g. air pressure, within the headset ear cup. The pressure on the external side of the seal verification module, e.g. atmospheric pressure, may remain substantially constant. The diaphragm may be deformable, e.g. have elastic properties. The diaphragm may be formed of an elasticated material. The diaphragm may be formed of an air tight material. The diaphragm may have a deformed configuration, e.g. a fully deformed configuration, and an undeformed configuration. The diaphragm may be biased towards the undeformed configuration. The diaphragm may assume its deformed, for example fully deformed, configuration in response to the pressure differential across the diaphragm reaching or exceeding the predetermined pressure differential. The predetermined pressure differential may be the pressure on the internal side of the seal verification module exceeding the pressure on the external side of the seal verification module by a predetermined amount. The diaphragm may assume its deformed configuration when the pressure on the internal side of the seal verification module changes, e.g. increase, by at least the predetermined amount. In use, the predetermined pressure differential may be a difference between a pressure within the headset ear cup when it is correctly situated around a user's ear to form an acoustic seal, and atmospheric pressure outside of the headset ear cup. The diaphragm may assume its undeformed configuration when the pressure differential across the diaphragm is balanced, e.g. the pressure within the headset ear cup is the same as the pressure outside of the headset ear cup in use. The diaphragm may be non-permeable. The diaphragm may be formed of an elastomer, for example Latex.

The pressure differential detector may further comprise an activation element. The activation element may be rigid. The activation element may be spaced apart from the diaphragm in its undeformed configuration. The activation element and the diaphragm may be relatively spaced such that when the diaphragm is in its fully deformed configuration the diaphragm is in contact with the activation element.

Collectively the diaphragm and the activation element may form a switch, for example a DC switch. The switch may form part of an electrical circuit with the indicator. The switch may break the electrical circuit when the diaphragm is in its undeformed configuration. The switch may complete the electrical circuit and therefore activate the indicator when the diaphragm is in its deformed configuration.

The diaphragm or the activation element may comprise one, two or more primary conducting elements. The primary conducting elements may be spaced apart. Each primary conducting element may be in the form of a conductive material forming a portion of said one of the diaphragm or activation element. Alternatively, each primary conducting element may be a pad formed of a conductive material, e.g. an electrical contact, provided on said one of the diaphragm or activation element. The conductive material may be flexible or rigid. The primary conducting elements may be spaced apart by an insulating element. The insulating element may be in the form or an insulating material, or an insulating panel. The insulating element may be located between the two or more spaced primary conducting elements such that an electrical current cannot flow directly between the primary conducting elements. The primary conducting elements may form part of the electrical circuit with the indicator. The space between the primary conducting elements may provide a break in the electrical circuit.

The other of the diaphragm or the activation element may comprise a secondary conducting element. The secondary conducting element may be in the form of a conductive material forming a portion of said one of the diaphragm or activation element. Alternatively, the secondary conducting element may be a pad formed of a conductive material, e.g. and electrical contact, provided on said one of the diaphragm or activation element. The conductive material may be flexible or rigid.

In use, when the diaphragm is in its deformed, for example fully deformed, configuration, the secondary conducting element may contact both/all of the primary conducting elements. The secondary conducting element may provide a conductive path between the primary conducting elements. The secondary conducting element may complete the electrical circuit and thus activate the indicator.

The seal verification module may further comprise a battery or other power source, for example a button or coin cell battery. The battery may be connected to/form part of the electrical circuit to provide power to the indicator. The battery may be removable from the seal verification module such that it can be replaced. Alternatively, the seal verification module may comprise a power connection arrangement. The power connection arrangement may be configured to connect to a battery external to the seal verification module.

The seal verification module may further comprise a battery state indicator. The battery state indicator may comprise a light indicator to provide visual indication of the level of charge in the battery. The light indicator may comprise an LED, in particular an RGB LED. The light indicator may comprise a plurality of LEDs. The LED(s) may comprise an associated resistor(s). The battery state indicator may comprise a display. Where the indicator for providing seal verification comprises a display, the battery state indicator may be comprised in the display for the seal verification indication. The battery state indicator may comprise a switch to provide selective indication of the battery state. The switch may be a momentary switch to provide selective momentary indication of the battery state. For example, a user may check the battery state via the switch prior to using the seal verification module to verify correct situation of a headset. Beneficially, the battery charge is conserved and maximised by the switch switching off the battery state indicator when it is not required.

The pressure differential detector may further comprise a vent port. The vent port may be configured to expose the diaphragm to the external side of the seal verification module. The vent port may be provided in the activation element.

The diaphragm and the activation element may form a chamber therebetween. The volume of the chamber may be lower when the diaphragm is in its deformed configuration compared to when the diaphragm is in its undeformed configuration. The vent port may provide a flow path from the chamber to the atmosphere, e.g. to the outside of the headset ear cup in use. The vent port may maintain a constant pressure in the chamber The vent port may maintain the chamber at atmospheric pressure. The vent port may permit deformation of the diaphragm.

The seal verification module may comprise a substrate to support any or all of the heretofore described components of the seal verification module. The substrate may be formed of plastic.

The pressure differential detector may further comprise a mounting element. The mounting element may be connected to the periphery of the diaphragm. The mounting element may have an annular form, e.g. may be in the form of a flange or a washer. The mounting element may be formed of a closed cell foam. The mounting element may be configured to mount the pressure differential detector in the substrate. In particular the mounting element may sealingly mount the pressure differential detector to the substrate. The mounting element may form a seal, e.g. an air seal, between the diaphragm and the substrate. Beneficially, this ensures that in use there is no air leakage path out of the ear cup around the pressure differential detector, in particular around the diaphragm.

According to a second aspect there is provided a headset comprising:
a headband configured to situate the headset on a user's head;
an ear cup mounted on the head band, for example at the end of the headband, and comprising an acoustic sealing element configured to form an acoustic seal around a user's ear;
wherein the ear cup is provided with a seal verification module, for example according to any embodiment of the first aspect, the seal verification module optionally configured to selectively verify the acoustic sealing element correctly situated around a user's ear to form an acoustic seal.

The internal side of the seal verification module may be proximal the inside of the ear cup. The external side of the seal verification module may be proximal the outside of the ear cup.

In use, a verification process can be carried out to determine whether an ear cup is correctly situated such that the acoustic sealing element forms an acoustic seal around a user's ear. The verification process involves an application of pressure, in particular a momentary application of pressure, on the outside of the ear cup, for example by a user momentarily pressing the outside of the ear cup. Herein, momentary and momentarily refer to approximately 1 second. A pressure in the region of 3440 Pa to 3460 Pa may be applied to the outside of the ear cup. Preferably a pressure of approximately 3450 Pa (0.500449335 psi) may be applied to the outside of the ear cup. If the ear cup is correctly situated such that the acoustic sealing element forms an acoustic seal around the user's ear, pressing the outside of the ear cup causes an increase in pressure within the ear cup such that the pressure differential across the diaphragm of the seal verification module is at least the predetermined pressure differential and the indicator of the seal verification module is activated. Thus, a user is provided with an indication verifying that the ear cup is correctly situated. If the ear cup is incorrectly situated such that the acoustic sealing element does not form an acoustic seal around the user's ear, pressing the outside of the ear cup causes no or insufficient pressure increase within the ear cup because air escapes between the acoustic sealing element and the user's head. Accordingly, the pressure differential across the diaphragm of the seal verification module is less than the predetermined pressure differential and the indicator of the seal verification module is not activated. Thus, the lack of indication informs the user that the ear cup is not correctly situated.

Beneficially, the headset may provide selective and immediate real-time verification of each ear cup being correctly situated such that the acoustic sealing element forms an acoustic seal around a user's ear.

The headband may be configured to secure the headset to a user's head. In particular, the headband may be configured to secure the at least one ear cup to a user's head. The headband may be configured to situate each ear cup around a user's ear. The headband may be adjustable by any suitable adjustment means known in the art. Beneficially this allows the headset to accommodate user's having varying size heads.

Each ear cup may be acoustically resistant. Each ear cup may comprise a rigid shell. The rigid shell may be formed of plastic, for example High Density Polyethylene. The rigid shell may be moulded. The rigid shell may be connected to the headband by any suitable connection means known in the art. The rigid shell may support the acoustic sealing element. The rigid shell may comprise a flange. The flange may support the acoustic sealing element. The acoustic sealing element may be compressible, for example the acoustic sealing element may have a compression indentation force of approximately 20-30%. Preferably, the acoustic sealing element may have a compression indentation force of approximately 25%. The predetermined pressure differential may depend on the compressibility of the acoustic sealing element. The predetermined pressure differential may be equivalent to a change of pressure inside the ear cup of approximately 3-7%. Preferably, the predetermined pressure differential may be equivalent to a change of pressure inside the ear cup of approximately 5%. The predetermined pressure differential may be in the region of 4000 Pa to 5000 Pa, preferably in the region of 4500 Pa to 5500 Pa, further preferably in the region of 5000 Pa to 5100 Pa. The acoustic sealing element may be a sound resistant cushion. The acoustic sealing element may be formed of acoustic foam, for example charcoal foam.

Each ear cup may comprise an acoustic damping element. The acoustic damping element may be supported inside the rigid shell. The acoustic damping element may be formed of, for example, acoustic foam. The acoustic foam may be charcoal foam. The acoustic foam may be covered in an air resistant material layer. The air resistant material layer may be formed of an air resistant protective hygienic material, for example plastic or a gel compound.

All or part of the seal verification module may be integral with the headset. Some or all of the seal verification module components may be integral with the ear cup. Some or all of the seal verification module components may be integral with the rigid shell. In this case, the substrate of the seal verification module may be the rigid shell of the ear cup. Alternatively, the seal verification module may be fitted to, mounted on, or recessed into the ear cup, in particular the rigid shell. The seal verification module may be recessed into the rigid shell. Beneficially, the seal verification module may be retrofitted to an existing headset. The substrate of the seal verification module may be formed of the same material as the rigid shell, for example plastic.

Where the indicator comprises a light indicator, the light indicator may be provided on the outside of the ear cup. Beneficially, this may allow verification by other personnel, for example a supervisor, health and safety, or manager in a workplace environment. Where the indicator comprises a sound indicator, the sound indicator may be provided on the inside of the seal verification module. Beneficially, this may allow the user to receive verification themselves.

Where the battery is external to the seal verification module, the battery may be fitted to, mounted on or recessed into the ear cup, in particular the rigid shell.

According to a third aspect there is provided a method of retrofitting a seal verification module according to the first aspect to a headset comprising an ear cup, the method comprising:
mounting the seal verification module to the ear cup.

The seal verification module may be mounted to the ear cup with the pressure differential detector exposed to both the internal side of the ear cup and the external side of the ear cup, such that the pressure differential detector may detect the pressure differential between the internal side of the ear cup and the external side of the ear cup. The pressure differential detector may be exposed to the external side of the ear cup via the vent port.

The ear cup may comprise a rigid shell. The seal verification module may be mounted on or recessed into the rigid shell.

Where the seal verification module does not comprise a battery, the method may further comprise connecting the seal verification module to a battery.

According to a fourth aspect there is provided a method of verifying that a headset comprising an ear cup is correctly situated against a surface such that the ear cup forms an acoustic seal against the surface, the method comprising:
pressing the ear cup against the surface;
detecting a pressure differential between an internal side of the ear cup and an external side of the ear cup when the ear cup is pressed against the surface; and
activating an indicator when the detected pressure differential reaches or exceeds a predetermined pressure differential associated with the ear cup being correctly situated.

Beneficially, the method provides real-time, in-situ, selective verification of the ear cup being correctly situated to form an acoustic seal.

The surface may be a user's head. The ear cup may form an acoustic seal around a user's ear.

The headset may be a headset according to the second aspect.

According to a fifth aspect there is provided a headset for providing hearing protection, the headset comprising:
two ear cups having a sound-resistant edge seal;
an adjustable headband; and
at least one electrical low-pressure diaphragm module, wherein the/each electrical low-pressure diaphragm module is retro fitted on the exterior or recessed into an outer layer of an associated one of said ear cups; wherein a momentary press on each respective ear cup while the headset is worn properly initiates the associated electrical ultra low-pressure diaphragm module which activates a sounder and light to indicate when the air pressure level within the ear cups is at a predetermined air pressure criteria of a good ear cup fit.

Beneficially, this construction permits accurate recognition of air pressure variances which may signify a breach of the acoustic seal.

The electrical low-pressure diaphragm module may be the earlier described pressure differential detector. The sounder and light may be comprised in the earlier described indicator.

At least two notification systems may be coupled to the electrical low-pressure diaphragm module to produce a notification to indicate when the air pressure level within the ear cups is at the predetermined air pressure criteria of a good headset fit.

The at least two notification systems may be comprised in the earlier described indicator. The at least two notification systems may comprise the sounder and the light.

The above summary is intended to be merely exemplary and non-limiting. It should be understood that features defined above in accordance with any aspect of the present disclosure or below relating to any specific embodiment of the disclosure may be utilized, either alone or in combination with any other defined feature, in any other aspect or embodiment or to form a further aspect or embodiment of the disclosure.

Features in one aspect may be applied as features in any other aspect in any appropriate combination. For example, apparatus features may be applied as method features and vice versa.

### BRIEF DESCRIPTION OF DRAWINGS

FIG. 1 is a perspective view of a headset;

FIG. 2 is a schematic internal view of a seal verification module of the headset of FIG. 1;

FIG. 3 is a schematic external view of the seal verification module of FIG. 2;

FIG. 4 is a schematic exploded view of a pressure differential detector of the seal verification module of FIG. 2;

FIG. 5a is a schematic assembled view of the pressure differential detector of FIG. 4 in a non-activated configuration;

FIG. 5b is a schematic assembled view of the pressure differential detector of FIG. 4 in an activated verification configuration;

FIG. 6a is a perspective view of an alternative headset; and

FIG. 6b is a schematic internal view of the seal verification module of the headset of FIG. 6a.

### DETAILED DESCRIPTION

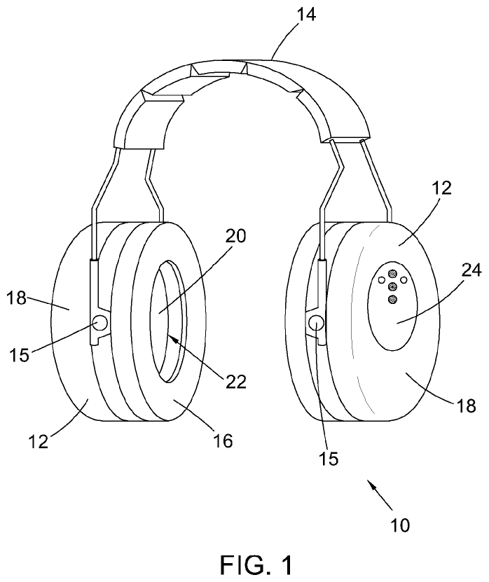

FIG. 1 shows a headset 10, e.g. an ear defender muff type headset. The headset comprises two ear cups 12 connected by a headband 14. In use, the ear cups 12 are positioned on either side of a user's head, each ear cup 12 covering one of the user's ears. The headband 14 extends across the top of the head between the ear cups 12. The headband 14 is adjustable so as to accommodate users of different head size. The ear cups 12 are mounted to either end of the headband 14 via hinges 15 such that each ear cup can be articulated relative to the headband 14 and therefore accommodate users of different head shape. Such headbands and ear cup mounting means are generally known and are beneficial in ensuring a good fit of the headset on a user's head, and in particular proper situation of the ear cups around a user's ears.

Each ear cup 12 comprises an acoustic sealing element 16. The acoustic sealing element 16 is formed of acoustic foam. The acoustic sealing element 16 is covered in a layer of air resistant protective hygienic material. In use the acoustic sealing element 16 forms an acoustic seal around a user's ear when the ear cup 12 is correctly situated. Each ear cup 12 further comprises a rigid shell 18. The rigid shell 18 is formed of plastic. Each ear cup 12 comprises an internal void 22 defined by the rigid shell 18. The rigid shell 18 comprises a flange (not shown) which supports the acoustic sealing element 16. The acoustic sealing element 16 defines the entrance to the void 22 within the ear cup 12. When the ear cup 12 is correctly situated around a user's ear, the user's head closes the void 22. The ear cup 12 further comprises an acoustic damping element in the form of an internal lining 20. The internal lining 20 is formed of acoustic foam. The internal lining 20 lines the inside of the rigid shell 18, only partially filling the void 22.

The headset 10 further comprises a seal verification module 24 mounted in one of the ear cups 12. The seal verification module 24 is removably mounted in the ear cup so that it may be repaired or replaced. In other embodiments, a seal verification module is retrofitted to a headset. In other embodiments, a seal verification module is mounted in each ear cup. Furthermore, in other embodiments the/each seal verification module is integral with the/each ear cup.

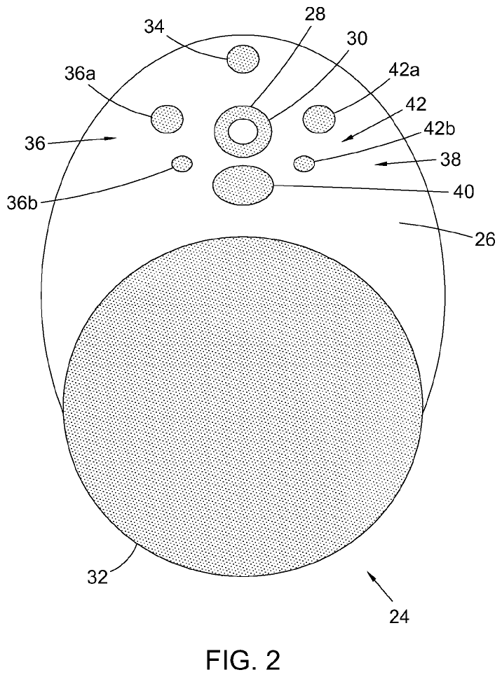

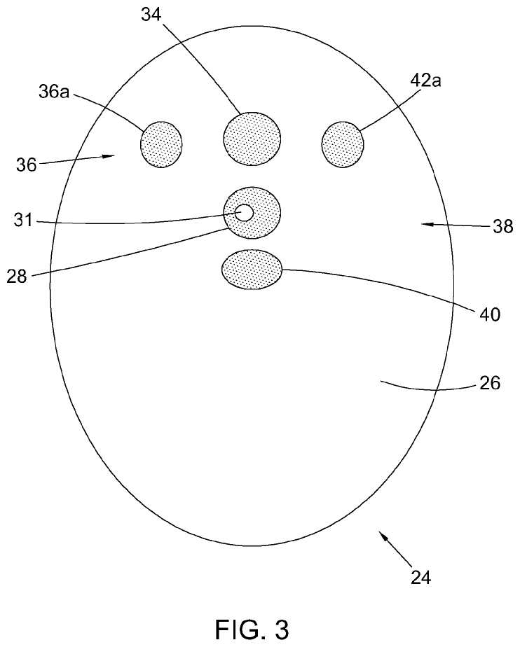

FIG. 2 shows a schematic of the seal verification module 24 as viewed from a side that is inside the ear cup 12 in use, herein referred to as the internal side. FIG. 3 shows a schematic of the seal verification module 24 as viewed from a side that is outside the ear cup 12 in use, herein referred to as the external side. The seal verification module 24 comprises a substrate 26 on which the components of the seal verification module 24 are mounted. The substrate 26 is formed of plastic. In particular, the substrate 26 is formed of the same plastic as the rigid shell 18. In alternative embodiments where the seal verification module is integral with the ear cup, the rigid shell acts as the substrate.

With continued reference to FIGS. 2 and 3, the seal verification module 24 comprises a battery 32 (see FIG. 2). The battery 32 is a coin cell battery, however in other embodiments any other suitable form of battery or power source may be used. The battery 32 is removable from the seal verification module 24 such that it can be replaced. The seal verification module 24 further comprises a switch 34 for selectively checking the state of the battery 32. The switch 34 is connected to a battery state indicator 36. The battery state indicator 36 provides an indication of the charge level of the battery 32 when the switch 34 is in an ‘on’ condition. The battery state indicator 36 comprises an LED 36a, in particular an RGB LED, however in other embodiments any indicator suitable for indicating the charge level of the battery may be utilised, for example a display or a series of LEDs. The LED 36a is visible from the external side of the seal verification module 24 (FIG. 3). The battery state indicator 36 further comprises a resistor 36b associated with the LED 36a.

The seal verification module 24 further comprises a pressure differential detector 28. The pressure differential detector 28 is configured to detect the pressure differential, e.g. air pressure differential, e.g. difference in pressure, between the internal side of the seal verification module 24 and the external side of the seal verification module 24. When implemented in headset 10, the external side of the seal verification module 24 is generally at atmospheric pressure and remains substantially constant. When implemented in headset 10, the internal side of the seal verification module 24 (e.g. the internal void 22 of the ear cup 12) is generally at atmospheric pressure, but is exposed to fluctuation in pressure during a verification process for determining whether the ear cup 12 is correctly situated such that the acoustic sealing element 16 forms an acoustic seal around a user's ear. The verification process involves pushing the ear cup 12 against the user's head by pressing the rigid shell 18 of the ear cup 12. Where the acoustic sealing element 16 is correctly situated around a user's ear to form an acoustic seal, the pressure within the void 22 will change during the verification process. The change in pressure in the internal void 22 when the ear cup 12 is correctly situated results in an ultra-low pressure differential across the pressure differential detector 28. The pressure differential detector 28 generally acts as a switch, in particular a DC switch, which is biased towards the ‘off’ condition and changes to the ‘on’ condition in response to the ultra-low pressure differential across the pressure differential detector 28 reaching or exceeding a predetermined level. The predetermined pressure differential is associated with the expected change in pressure within the void 22 when the ear cup 12 is correctly situated. The predetermined pressure differential across the pressure differential detector 28 is achieved when the pressure within the void 22 increases by a predetermined amount. The pressure external to the ear cup remains substantially constant. Thus the pressure differential detector 28 functioning as a switch changes from the ‘off’ condition to the ‘on’ condition when the pressure within the void 22 increases by at least a predetermined amount. Where the acoustic sealing element 16 is not correctly situated around a user's ear to form an acoustic seal, air will leak from the internal void 22 between the acoustic sealing element 16 and the user's head during the verification process and the pressure within the void 22 will not change, e.g. will remain approximately constant. The pressure differential detector 28 will thus remain in the ‘off’ condition when the ear cup is not correctly situated.

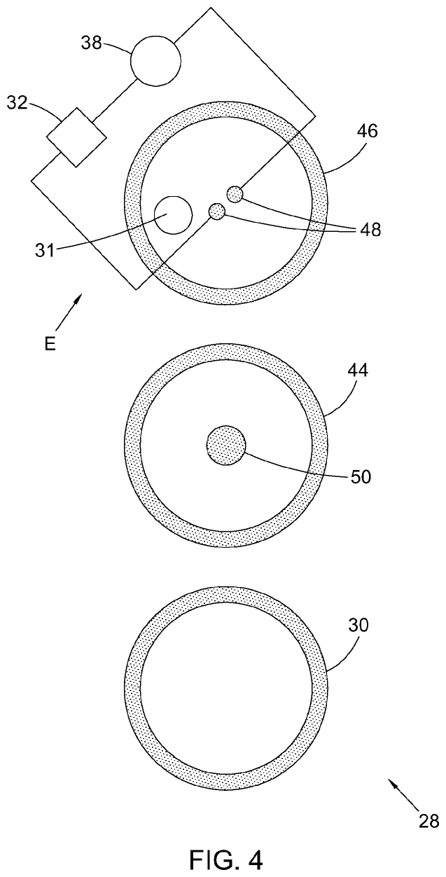

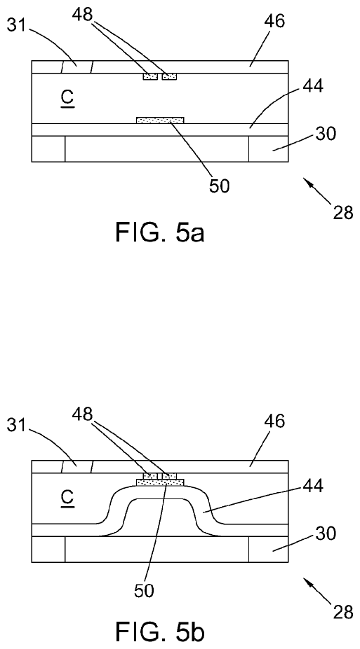

As can be seen in FIG. 2, the pressure differential detector 28 comprises a mounting element 30 for mounting the pressure differential detector 28 in the substrate 26. In particular, the mounting element 30 sealingly mounts the pressure differential detector 28 in the substrate 26 such that air cannot flow between the pressure differential detector 28 and the substrate 26, e.g. around the sides of the pressure differential detector 28. The mounting element 30 is formed of a closed cell foam. As can be seen in FIG. 3, the pressure differential detector 28 further comprises a vent port 31 to the external side of the seal verification module 24. Further details of the pressure differential detector 28, and the features providing the switching functionality, are later described with reference to FIGS. 4, 5a and 5b.

With continued reference to FIGS. 2 and 3, the seal verification module 24 further comprises an indicator 38. The indicator 38 forms part of an electrical circuit E which also comprises the battery 32 and the pressure differential detector 28. The indicator 38 is connected to the pressure differential detector 28 such that when the pressure differential detector 28 is in the ‘on’ condition the indicator 38 is activated. The indicator 38 comprises a sound indicator 40 in the form of a buzzer. The sound indicator 38 directs sound towards the internal side of the seal verification module 24 such that in use it can be heard by a person wearing the headset 10. The indicator 38 further comprises a light indicator 42. The light indicator 42 comprises an LED 42a and a resistor 42b associated with the LED. The LED 42a is visible on the external side of the seal verification module 24 such that a person not wearing the headset 10 can see the LED, for example a supervisor or manager in a workplace environment. In other embodiments the seal verification module may comprise only a sound indicator or only a light indicator. In other embodiments the sound indicator may be any other known form of sound indicator, and/or the light indicator may be any other known form of light indicator. In other embodiments the seal verification module may comprise, alternatively or in addition, any other known form of indicator.

FIG. 4 shows an exploded schematic view of the pressure differential detector 28 of the seal verification module 24. FIGS. 5a and 5b are schematic assembled views through a cross section of the pressure differential detector 28. The pressure differential detector 28 comprises three layers. As described above, the pressure differential detector 28 comprises a mounting element 30. The mounting element 30 is the first layer. The mounting element 30 is the layer towards the internal side of the seal verification module 24. The mounting element 30 is annular in shape. The ultra-low pressure differential detector 28 comprises a diaphragm 44. The diaphragm 44 is the second layer. The diaphragm 44 is the middle layer. The mounting element 30 supports the periphery of the diaphragm 44. The diaphragm 44 is deformable between an undeformed configuration (FIG. 5a) and a fully deformed configuration (FIG. 5b). The diaphragm 44 is biased towards the undeformed configuration. One side of the diaphragm 44 is exposed to the internal side of the seal verification module 24. The other side of the diaphragm 44 is exposed to the other side of the seal verification module 24. Thus a pressure differential across the seal verification module 24 (and between the inside and outside of the ear cup 12 in use) is experienced by the diaphragm 44. The diaphragm 44 assumes its fully deformed configuration when the pressure differential across the diaphragm 44 meets or exceeds the predetermined pressure differential associated with the ear cup 12 being correctly situated around a user's ear so as to form an acoustic seal.

The pressure differential detector 28 further comprises an activation element 46. The activation element 46 is the third layer. The activation element 46 is the layer towards the external side of the seal verification module 24. The activation element 46 comprises two primary conducting elements 48 in the form of electrical contacts. The primary conducting elements 48 are spaced apart such that electrical current cannot flow directly between them. The primary conducting elements 48 form part of the electrical circuit E with the battery 32 and the indicator 38 (see FIG. 4). The space between the primary conducting elements 48 provides a break in the electrical circuit. The diaphragm 44 comprises a single secondary conducting element 50 in the form of an electrical contact. The activation element 46 is arranged relative to the diaphragm 44 such that when the diaphragm 44 is in the undeformed configuration the secondary conducting element 50 is spaced apart from the primary conducting elements 48, and when the diaphragm is in the fully deformed configuration the secondary conducting element 50 is in contact with both of the primary conducting elements 48 to provide a conductive path between the two primary conducting elements 48. The secondary conducting element 50 completes the electrical circuit E when the diaphragm 44 is in the fully deformed configuration and thus activates the indicator 38. In this way, the pressure differential detector 28 acts as a switch in the electrical circuit E.

As can be seen in FIGS. 5a and 5b, the diaphragm 44 and the activation element 46 form a chamber C therebetween. The volume of the chamber C changes as the diaphragm 44 deforms, therefore to maintain the chamber at a constant pressure (the pressure at the external side of the seal verification module 14, e.g. atmospheric pressure) the activation element 46 is provided with the aforementioned vent port 31. The vent port 31 extends between the chamber C and the external side of the seal verification module 14.

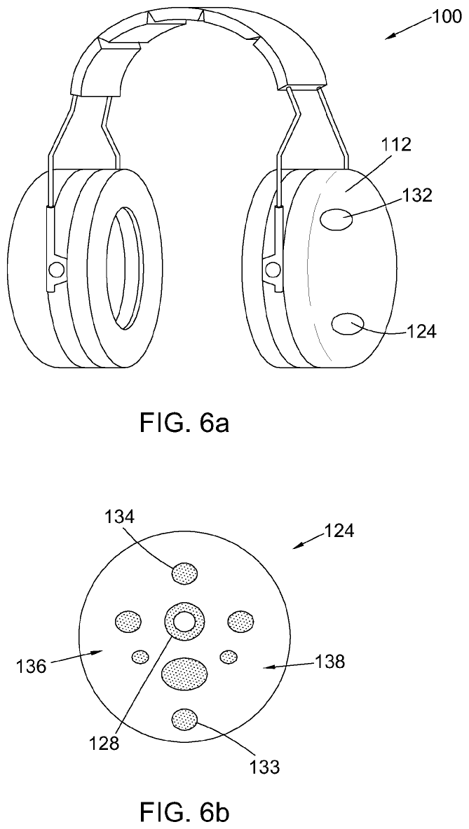

FIGS. 6a and 6b provide an alternative headset 100 and seal verification module 124. Like features are provided with like reference numerals to the embodiment of FIGS. 1 to 5, augmented by 100. The headset 100 and seal verification module 124 substantially correspond to headset 10 and seal verification module 24, with the exception that the battery 132 is provided separately to the seal verification module 124, as can be seen in FIG. 6a. The battery 132 and the seal verification module 124 are mounted in the ear cup 112 separately. As shown in FIG. 6b, the seal verification module 124 comprises a power connection arrangement 133. The power connection arrangement 133 is comprised in an electrical circuit with the indicator 138 and the pressure differential detector 128. The power connection arrangement 133 is configured to provide an electrical connection between the seal verification module 124 and the battery 132. The battery state indicator 136 and switch 134 are also electrically connected to the battery 132 via the power connection arrangement 133. The battery state indicator may additionally or alternatively provide verification of the electrical connection between the seal verification module and the battery.

Whilst certain embodiments are described, these embodiments have been presented by way of example only, and are not intended to limit the scope of the invention. Furthermore, various omissions, substitutions and changes in the form of the embodiments described herein may be made without departing from the scope of the invention.

1. A seal verification module for a headset ear cup, the module comprising:
an indicator configured to provide an indication in response to being activated; and
a pressure differential detector configured to detect a pressure differential between an internal side of the seal verification module and an external side of the seal verification module, and configured to activate the indicator in response to the detected pressure differential reaching or exceeding a predetermined pressure differential associated with verifying the headset ear cup being correctly situated to form an acoustic seal against a surface.

2. The seal verification module of claim 1, wherein the predetermined pressure differential corresponds to the pressure on the internal side of the seal verification module increasing by a predetermined amount.

3. The seal verification module of claim 1, wherein the pressure differential detector comprises a diaphragm having one side exposed to the internal side of the seal verification module and another side exposed to the external side of the seal verification module such that a pressure differential across the diaphragm is the pressure differential between the internal side of the seal verification module and the external side of the seal verification module

4. The seal verification module of claim 3, wherein the diaphragm has a deformed configuration in which the indicator is activated and an undeformed configuration in which the indicator is not activated, wherein the diaphragm is configured to assume the deformed configuration in response to the pressure differential across the diaphragm reaching or exceeding the predetermined pressure differential.

5. The seal verification module of claim 3, wherein the pressure differential detector further comprises an activation element, and wherein the activation element and the diaphragm are configured to collectively act as a switch to activate the indicator when the diaphragm is in its deformed configuration.

6. The seal verification module of claim 5, wherein one of the diaphragm and the activation element comprises two or more primary conducting elements, wherein the other of the diaphragm and the activation element comprises a secondary conducting element, and wherein when the diaphragm is in its deformed configuration the secondary conducting element provides a conductive path between the primary conducting elements to activate the indicator.

7. The seal verification module of claim 5, wherein the pressure differential detector further comprises a chamber formed between the diaphragm and the activation element, and wherein the chamber comprises a vent port.

8. The seal verification module of any claim 1, further comprising a substrate supporting the indicator and the ultra-low pressure differential detector.

9. The seal verification module of claim 8, wherein the pressure differential detector further comprises a flange, wherein the flange sealingly mounts the pressure differential detector on the substrate.

10. The seal verification module of claim 1, further comprising a battery.

11. The seal verification module of any claim 1, wherein the indicator comprises a light indicator and/or a sound indicator.

12. A headset comprising:
a headband configured to situate the headset on a user's head;
an ear cup, each ear cup mounted on the headband, for example on an end of the headband, and comprising an acoustic sealing element configured to form an acoustic seal around the user's ear;
wherein the ear cup is provided with a seal verification module of any preceding claim configured to selectively verify the acoustic sealing element correctly situated around the user's ear to form an acoustic seal.

13. The headset of claim 12, wherein the head band is adjustable.

14. The headset of claim 12, wherein each ear cup comprises a rigid shell and wherein the respective seal verification module is integral to, recessed into or mounted in/on the rigid shell.

15. The headset of any of claim 12, wherein each ear cup comprises an acoustic damping element.

16. A method of retrofitting a seal verification module according to any of claim 1 to a headset comprising an ear cup, the method comprising:
mounting the seal verification module to the ear cup.

17. A method of verifying that a headset comprising an ear cup is correctly situated on a surface such that the ear cup forms an acoustic seal against the surface, the method comprising:
pressing the ear cup against the surface;
detecting a pressure differential between an internal side of the ear cup and an external side of the ear cup when the ear cup is pressed against the surface; and
activating an indicator in response to the detected pressure differential reaching or exceeding a predetermined pressure differential associated with the ear cup being correctly situated.

18. (canceled)

19. The method of claim 17, wherein pressing the ear cup against the surface comprises increasing the pressure within the ear cup when the ear cup is correctly situated, and wherein the predetermined pressure differential corresponds to the pressure within the ear cup increasing by a predetermined amount.
